(19) United States
(12) Patent Application Publication
(10) Pub. No.: US 2018/0060515 A1
(43) Pub. Date: Mar. 1, 2018

(54) AMS_BioRhythms computing program

(71) Applicant: RONALD LESLIE MONARCH, WRIGHTSTOWN, WI (US)

(72) Inventor: RONALD LESLIE MONARCH, WRIGHTSTOWN, WI (US)

(21) Appl. No.: 15/330,259

(22) Filed: Aug. 31, 2016

(51) Int. Cl. G06F 19/00; A61B 5/00; G09B 5/12; G09B 19/00

(52) U.S. Cl. CPC G06F 19/3406; G09B 19/00; G09B 5/12; A61B 5/4857

(57) ABSTRACT

This AMS_BioRhythms computing program is capable of operating on any computing device, tool and/or equipment with a computing based Operating System to calculate and determine the three major personal Bio-Rhythm cycles for any specific entity or any situation for any time period. Such computing device, etc., can, and will, include any computer PC, laptop, tablet, phone and/or other device capable of running any type of computing program, such devices with a monitor/screen used to produce information for any entity and/or situation, via home computer, internet or any place of business. This Bio-Rhythm computing program is capable of producing, for any entity or situation, a combination of any two entities and/or situations; any of a single daily or a single yearly Bio-Rhythm graphics, or double daily or double yearly Bio-Rhythm graphics. Said Bio-Rhythm graphics and information is based upon the entity's birthdate or any situation's inception/start date.

### OATH OR DECLARATION

See accompanying Oath/Declaration form!

### STATEMENT REGARDING FEDERALLY SPONSORED RESEARCH OR DEVELOPMENT

Not Applicable!

### BACKGROUND OF THE INVENTION

01 This author and inventor, Ronald L. Monarch DBA Astro Metaphysical Services, is hereinafter referred to as either “Monarch”, “Ronald L. Monarch” or “Ronald L. Monarch DBA Astro Metaphysical Services”.

02 The field of endeavor to which the invention of this AMS_BioRhythms computing program does pertain is computer programs, more specifically the science of life studies, and even more specifically Bio-Rhythm cycles and the manner in which such cycles pertain to the life cycle of any entity. Monarch has found that such do affect all forms of entities including people, critters of all types, countries and even any specific situation that we, as humans, tend to think of as not having any life cycle.

03 After reading Bernard Gittleson's book “BIO-RHYTHM A PERSONAL SCIENCE” and Vincent Mallardi's book “BIOCYCLES” in the mid 1980's I, “Monarch” became very intrigued by the idea of Bio-Rhythms being used as another tool to look at, and delve into, the mechanisms of any person's, or other entity's, live/s and the manner in which any such entity got involved with various other entities and situations in such entity's life.

04 Both Gittelson and Mallardi did use a system of calculations, or lookups, that an individual would work through, by hand, in order to achieve a very minute idea of the position of each of the “Physical”, “Emotional” and/or “Intellectual” cycles of any individual for any specific day. The idea of using such calculations and lookups did appear to Monarch to be quite cumbersome and difficult for the average world individual to use. Also, the idea of using such calculations and lookups for an accurate and more complete Bio-Rhythm cycle analysis did appear to Monarch to be quite insufficient for a more in depth study and analysis of the manner in which said Bio-Rhythm cycles do actually coincide with, and possibly influence, any type of relationship/s any entity might be involved with during a specific entity's life time.

05 Evidently, after Gittelson, Mallardi and others did take a brief “scientific” look at Bio-Rhythm cycles, such individuals, etc., came to the conclusion that there was not, nor is there, any evidence of any predictive value for eliminating, or reducing, accidents to be found under the Bio-Rhythmic scope. Also, the general discussion of Bio-Rhythm cycles and their effect/s on any individual's life or existence, especially within the “scientific” community, has heretofore centered more around the idea of negating any effect that said Bio-Rhythm cycles supposedly have upon any individual's existence.

06 As Ronald L. Monarch was, at the time, involved in learning computer programming, Monarch did decided to build a computer program capable of producing the Bio-Rhythm cycles in a more easily understood graphics format for any individual for any period in an individual's lifetime.

07 In the process of developing such a computer program Monarch did also improve Bio-Rhythm programing;
A) By producing a long and short screen version of the graphic representation.
B) By producing the Bio-Rhythm cycles graphics for each entity with more distinctive graphic representations for each of the physical, emotional and intellectual cycle representations.
C) By developing and introducing the idea of embedding the Bio-Rhythm cycles of a second entity, or situation, directly onto the Bio-Rhythm cycles graphic as produced for the first entity.
D) By Monarch's development and introduction of the idea of the single entity and double entity yearly Bio-Rhythm cycles and graphic representations. Monarch has found that the “yearly” Bio-Rhythm cycles graphic tends to bring more clarity of explanation as to why any specific daily situation in the lifetime of any specific entity did occur and why the entity did react in the manner that said entity did react to any specific situation on any one day, month or year.

09 This early version of this AMS_BioRhythms computing program was initially developed in “DOS” format for the use, study of and furtherance of the Bio-Rhythm cycles by this author and inventor, Ronald L. Monarch DBA Astro Metaphysical Services. Said DOS version of this AMS_BioRhythms computing program was also put forth to the public several years ago by this author and inventor, Ronald L. Monarch DBA Astro Metaphysical Services.

10 Monarch has now redeveloped such programming in the form of more modern programming tools and techniques so that such AMS_BioRhythms computing program can now be implemented for use on and via various popular computing devices and operating systems.

11 Heretofore, with the exception of this version of the AMS_BioRhythms computing program, there has been no other Bio-Rhythm cycles computing program, or any other format of Bio-Rhythm cycles production, that have even come close to the ideas and graphic representations that this inventor, Ronald L. Monarch, has developed and put forth. The only exception to this statement, of course, is Ronald L. Monarch's previous “DOS” version of the Bio-Rhythm cycles program.

12 Therefore, Ronald L. Monarch DBA Astro Metaphysical Services is the first, and only, individual or entity to apply to the USPTO for a patent concerning the scope of such information, graphics representations and ideas, etc., as described throughout the entirety of this application for a United States Patent, and as put forth and promoted by Ronald L. Monarch DBA Astro Metaphysical Services and this AMS_BioRhythms computing program.

13 Ronald L. Monarch DBA Astro Metaphysical Services has found that Bernard Gittelson, Vincent Mallardi and others that attempt to speak to the operation of Bio-Rhythm cycles are incorrect in their conclusions concerning Bio-Rhythm cycle as these “scientific” individuals focus only upon the entity and the day of any situation upon which they wish to focus.

14 Through extensive research with the use of this AMS_BioRhythms computing program, Ronald L. Monarch has come to the conclusion that Gittelson, Mallardi and other Bio-Rhythm researchers have focused too much upon a relatively narrow portion of an entity's life cycle. With their narrow focus on the Bio-Rhythm cycles of one entity at a time Gittelson, Mallardi and other Bio-Rhythm researchers have apparently failed to understand the manner in which one entity is likely to interact with any other type of entity.

15 This AMS BioRhythms computing program is meant to assist, more efficiently and effectively, in the study of the manner in which Bio-Rhythms actually affect the life time of any human, animal, business, country or any other entity or situation and the manner in which any such entity is likely to interact with any other entity or situation.

16 To this end, this program can be used as a “Person of Interest” and/or “Pre-Crime” tool! This inventor has, for the past several years, conceived ideas on the manner of such “Person of Interest” and/or “Pre-Crime” use. The database of such would likely be many times larger than the program itself. Through this inventor's use of and study using this AMS_BioRhythms computing program, this inventor has seen how this program can be used in the questioning of any “suspect” in connection with any situation that said “suspect” may have been involved. This inventor has given lectures and demonstrations on the manner in which this computer program can be used.

17 Ronald L. Monarch, the inventor of this AMS_BioRhythms computing program, does see that the next step in the programming of the Bio-Rhythm cycles would be to include a more adequate interpretation for any day or month that the program user would wish to have interpreted for any specific entity.

18 More specifics are coming forth in a book that Monarch has begun writing on the subject of Bio-Rhythms, as Monarch has come to understand Bio-Rhythm cycles and the influence such cycles are capable of exerting upon any entity's existence. In such book Monarch will explain Bio-Rhythms at a much deeper level than has, heretofore, been exhibited by Bernard Gittelson, Vincent Mallardi or any other individual or other entity that has worked with Bio-Rhythm cycles.

### SUMMARY OF THE INVENTION

19 The object of this invention is to promote a deeper and much improved understanding of Bio-Rhythm cycles and the manner in which such cycles are able to describe situations from the past, in the present and into the future lifetime of any entity or situation while supplying indications of the best manner of resolving any situation.

20 Once the computer program is opened, this computer program accepts, for analysis, a name the user wishes to use and a birthdate, or startdate, of any entity or situation.

21 This AMS_BioRhythms computing program then works through a series of calculations after which this program produces a graphic output onto any monitor screen one of the four possible main Bio-Rhythm graphics as has been requested by the user of this computer program. Such graphic output will allow the user to easily move the graphic representation either forward or backward in time. The user will also be able to capture, print or otherwise save such Bio-Rhythm cycles graphic

22 The resulting screen image will be either a daily or yearly graphic representation for a single entity, or a daily or yearly graphic representation for the set of Bio-Rhythms of two entities, with the Bio-Rhythms of the second entity imposed directly upon the chart of the Bio-Rhythms of the first entity as is directed by the program user.

23 This computer program is able to produce a compatibility rating, by percentage of compatibility, for any relationship at which the user would decide to look.

24 When the user finishes the user's work, the user may choose to close the program.

### BRIEF DESCRIPTION OF THE SEVERAL VIEWS OF THE DRAWINGS

25 The patent or application file contains at least one drawing executed in color. Copies of this patent or patent application publication with color drawing(s) will be provided by the Office upon request and payment of the necessary fee.

26 Of course, the actual screen print drawings, as produced to the monitor of the device used, will automatically be in color. The graphic top, middle and bottom lines will appear as a green color. Each of the Bio-Rhythms cycles depicted to the monitor/screen will appear as a red color for the first entity in all Bio-Rhythm cycles graphic representations. Each of the Bio-Rhythms cycles depicted to the monitor/screen will appear as a green color for the second entity in the double daily or double yearly graphic representations. Such colors are applied for a more distinctive depiction of each of the two entity's Bio-Rhythm cycles upon the same graphic representation.

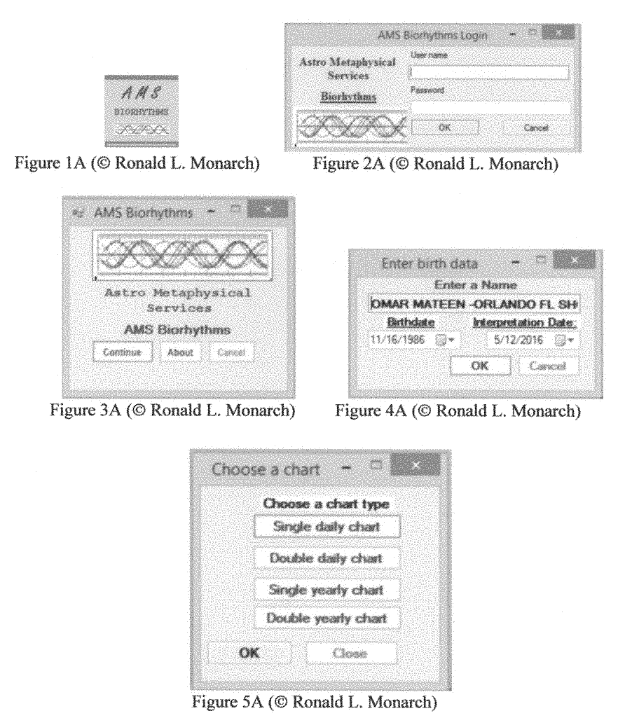

27 The FIG. 1A of the patent application drawings for this AMS_BioRhythms computing program is a desktop graphic icon of this AMS_BioRhythms computing program that would be used to start this AMS_BioRhythms computing program.

28 The FIG. 2A of the patent application drawings for this AMS_BioRhythms computing program is the graphic representation of the user Log-In frame for this AMS_BioRhythms computing program.

29 The FIG. 3A of the patent application drawings for this AMS_BioRhythms computing program is the graphic representation of the AMS_BioRhythms computing program start frame.

30 The FIG. 4A of the patent application drawings for this AMS_BioRhythms computing program is the graphic representation of the data entry frame for this AMS_BioRhythms computing program.

31 The FIG. 5A of the patent application drawings for this AMS_BioRhythms computing program is the graphic representation of the graphic chart option menu for this AMS_BioRhythms computing program.

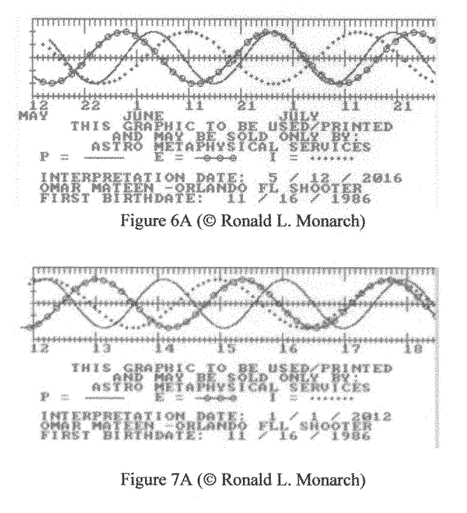

32 The FIG. 6A graphic of “INTERPRETATION DATE: 5/12/2016” does demonstrate a more easily distinguishable and understood graphical representation of the “P”—physical, “E”—emotional and “I”—intellectual cycles as portrayed for the daily Bio-Rhythm cycles graphics and the manner in which they interact and point to specific points of interest in any single entity's lifetime! Such new representation of each cycle was developed, and copyrighted, several years ago by Ronald L. Monarch as owner/operator of Astro Metaphysical Services.

33 The FIG. 7A graphic of “INTERPRETATION DATE: 1/1/2012” does demonstrate the single yearly Bio-Rhythm cycles graphic of the individual's yearly biorythm cycles as invented and developed several years ago through the research of Ronald L. Monarch as owner/operator of Astro Metaphysical Services. Such graphic does point to specific points of interest throughout each of the years listed for the entity. Ronald L. Monarch is of the belief that no other individual/other entity has ever produced such a representation, nor, prior to Ronald L. Monarch's representation of such biorythm cycles, has there ever been any individual/other entity that understands such a concept as the “yearly biorythm cycle”. Ronald L. Monarch as owner/operator of Astro Metaphysical Services has been writing, and continues to write, a more definitive book on a more adequate understanding of biorhythm cycles!

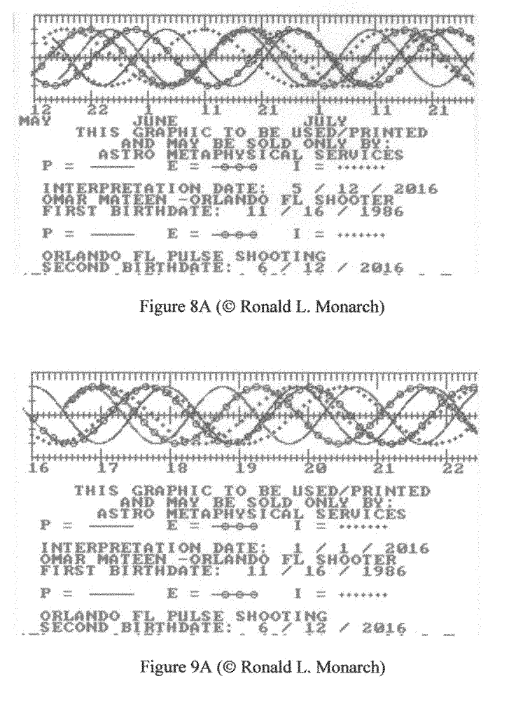

34 The FIG. 8A graphic of “INTERPRETATION DATE: 5/12/2016” does demonstrate the double daily graphic bio-rhythm cycles representation with the Orlando, Fla. Pulse Club shooting bio-rhythm cycles laid directly over the daily biorythm cycles for Omar Mateen, the apparent shooter during said incident. The graphic representation does begin on May 12, 2016 depicting each cycle as each cycle would appear beginning May 12, 2016 and continues forward. Such graphic representation does depict the various probable connections, and points of interest, between the entity and the situation in which the entity was involved while depicting the levels of each cycle for each entity/situation throughout the period shown. Each entity's/situation's individual bio-rhythm cycles have their own distinct colors for a more clear image of the manner in which the entities are likely to react with each other.

35 The FIG. 9A graphic of “INTERPRETATION DATE: 1/1/2016” does demonstrate the double yearly graphic bio-rhythm cycles representation with the Orlando, Fla. Pulse Club shooting yearly biorythm cycles laid over the yearly bio-rhythm cycles for Omar Mateen, the apparent shooter during the situation. Each cycle begins as each cycle would appear on 01/01/2016 and continues forward. Such graphic representation, as developed several years ago through the research and development of Ronald L. Monarch as owner/operator of Astro Metaphysical Services, depicts the various probable connections, and points of interest, on a monthly basis, between Omar Mateen and the Orlando, Fla. Pulse shooting incident, as separate and distinct entities. The individual bio-rhythm cycles of each of Omar Mateen and the actual shooting incident have their own distinct colors for a clear image of the manner in which each entity's bio-rhythm cycles are likely to react with each other.

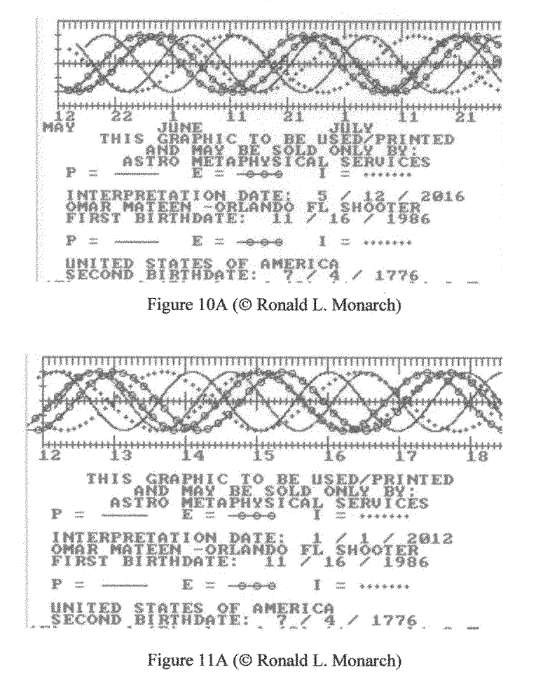

36 The FIG. 10A graphic of “INTERPRETATION DATE: 5/12/2016” does demonstrate the double daily graphic bio-rhythm cycles representation with the USA's daily bio-rhythm cycles laid directly over the daily bio-rhythm cycles for Omar Mateen, the apparent shooter during the Orlando, Fla. Pulse Club incident. Such cycles graphic does begin on May 12, 2016 depicting each cycle as each would appear on May 12, 2016 and continues forward. Such graphic representation does depict the various probable connections, and points of interest, on a monthly basis, between Omar Mateen and the USA while denoting the levels of each cycle for each individual throughout the period graphed.

37 The FIG. 11A graphic of “INTERPRETATION DATE: 1/1/2012” does demonstrate the double yearly graphic bio-rhythm cycles representation with the yearly United States of America bio-rhythm cycles laid directly over the yearly biorythm cycles for Omar Mateen, the apparent shooter of the Orlando, Fla. Pulse Club incident. Each cycle begins as each cycle would appear on 01/01/2012 and continues forward. Such graphic representation does depict the various probable connections, and points of interest, between Omar Mateen and the United States of America throughout the years as depicted.

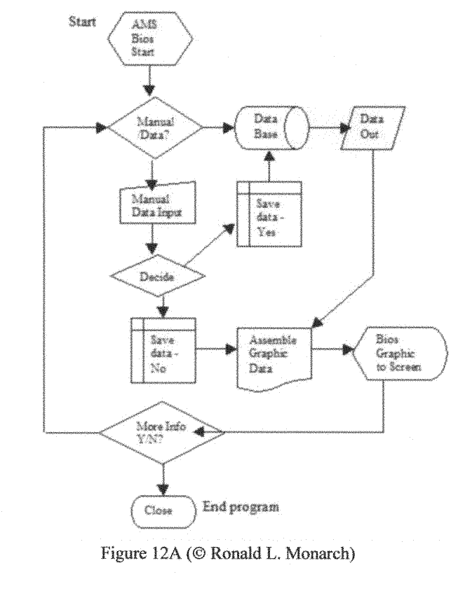

38 The FIG. 12A graphic is a graphic diagram of the flowchart for the basic manner in which this AMS_BioRhythms computing program does operate.

### DETAILED DESCRIPTION OF THE INVENTION

39 With the construction and production of said “AMS_BioRhythms—A computer program to be implemented on any PC, Laptop, Tablet, Phone and/or any other computing device” Monarch has given each of the “Physical”, “Emotional” and “Intellectual” cycles their own distinctive graphic representation producing said graphic with a more distinct look so as to make the daily Bio-Rhythm compilation more easily and quickly read and understood at a quick glance.

40 Using this Bio-Rhythm computer program as developed by Ronald L. Monarch DBA Astro Metaphysical Services, Monarch did begin by analyzing Monarch's life and Monarch's interactions with other individuals and life situations. After analyzing Monarch's life through these Bio-Rhythms Monarch soon began looking at the Bio-Rhythms of other individuals that were involved in Monarch's life. Monarch soon began looking at separate printed versions of each individual's single daily Bio-Rhythms and analyzing the manner in which they did/did not appear to coordinate with each other and looking at each entity's situations as they appeared between the two entities in question that Monarch did attempt to analyze.

41 Even through the analysis of Monarch's single daily Bio-Rhythms, and the single daily Bio-Rhythms of others, Monarch began to see that there was more to the Bio-Rhythms cycles than previously reported by other Bio-Rhythm analysts and what such Bio-Rhythm cycles could tell us about our lives and the lives of others. Monarch did begin to understand that the Bio-Rhythm cycles could tell more about any individual, any other entity and any life situation than had previously been put forth by Gittelson or any other Bio-Rythm analyst. The more Monarch did study Bio-Rhythms the more Monarch became convinced that there is actually more information that Bio-Rhythms have to give us than anyone had previously proposed. Monarch has found this to be true even using the single daily Bio-Rhythm graphic representation.

42 Since there appeared to be at least a bit of guess work in trying to determine when and where each cycle of one entity might coincide with one, or more, cycle/s of the second entity in question. Such analysis, also, took too much time to decide whether, and in what manner, any cycle, or cycles of one entity were in conjunction with any cycle, or cycles of the second entity.

43 Monarch's next step, then, was to re-program Monarch's Bio-Rhythm computer program so that said program would print a second individual's, or other entity's, Bio-Rhythm graphic representation directly upon the graphic of the first entity's Bio-Rhythm graphic representation. Such superimposition of one entity's Bio-Rhythm cycles directly over any other entity's Bio-Rhythms cycles did make for a quicker, and more accurate analysis of the manner of interaction between the two entities!

44 After the printing of the Bio-Rhythms cycles of one entity directly upon the same graphic Bio-Rhythms cycles of another entity, Monarch then asked the question of “Whether the Bio-Rhythm cycles could be produced for an accurate representation of the Bio-Rhythm cycles across any period of years for any entity?” A second question then asked by Monarch was “What would such ‘yearly’ Bio-Rhythm cycles look like?”

45 Therefore, within about one to two months Monarch did develop the “yearly” Bio-Rhythm cycles so that said cycles did coordinate well, on a more broad basis, with any entity's life situations that did, or will, arise in the entity's lifetime. These single “yearly” Bio-Rhythm cycles did also prove to coordinate well with the entity's months and days as described within any entity's single daily cycle and double daily Bio-Rhythm cycles.

46 While a preferred embodiment of the present invention has been illustrated for desirability of its specific details and has specific preferred examples of the use of this invention, and the graphic representations and output of this invention, further embodiments, modifications and variations are contemplated and embedded within this AMS_BioRhythms computing program, all according to the spirit and scope of the encapsulated claims of this AMS_BioRhythms computing program patent application.

47 Any person of ordinary skill capable of operating any computing based device and data entry could make and use this AMS_BioRhythms computing program invention without extensive experimentation in order to produce the graphic representations, as described within the “Brief Description of the Several Views of the Drawings” portion of this herein United State Patent Office Patent application document.

48 Such operation of this AMS_BioRhythms computing program would proceed in the following manner;
48-a) The user would click the onscreen graphic icon (FIG. 1A of the patent application drawings for this AMS_BioRhythms computing program patent application drawings) to launch this AMS_BioRhythms computing program.

48-b) The user then enters the user's name and password into the AMS_BioRhythms Log-in frame (FIG. 2A of the patent application drawings for this AMS_BioRhythms computing program). Clicking or touching the “Cancel” button will close the program.
48-c) If the user clicks or touches the “OK” button after entering the proper name and password the user is the shown the main program start frame. At this point the user is able to choose to “Cancel” the program, learn about Bio-Rhythms and the capabilities of this AMS_BioRhythms computing program and/or “Continue” on to use this AMS_BioRhythms computing program.
48-d) By choosing to “Continue”, this AMS_BioRhythms computing program produces, to the screen, the data entry frame (FIG. 4A of the patent application drawings for this AMS_BioRhythms computing program) where the user enters an appropriate name and birthdate/startdate which the user would like to research.
48-e) The user may use the date set in the “Interpretation date” box (also seen in FIG. 4A), to determine the date on which the Bio-Rhythm cycles graphic will actually begin, or the user may set any other start date in said “Interpretation date” box.
48-f) The user, then, either clicks the “Cancel” button to close the program, or clicks the “OK” button to continue on to the Bio-Rhythms chart selection frame (FIG. 5A of the patent application drawings for this AMS_BioRhythms computing program) where the user is able to choose to do a single or double daily, or a single or double yearly Bio-Rhythm cycles graphic.
48-g) If the user has chosen either a single daily, or single yearly Bio-Rhythm graphic, this program proceeds to produce, on the monitor, the requested single daily graphic (as demonstrated by FIG. 6A of the patent application drawings for this AMS_BioRhythms computing program) or single yearly graphic (as demonstrated by FIG. 7A of the patent application drawings for this AMS_BioRhythms computing program) as requested by the user.
48-h) If the user has chosen either a double daily, or double yearly bio-rhythm graphic, the user will be prompted to enter the name of any second entity/situation and the birthdate/startdate of said second entity/situation.
48-i) This program then proceeds to produce, on the monitor, the requested double daily graphic (as demonstrated by FIG. 8A of the patent application drawings for this AMS BioRhythms program) or double yearly graphic (as demonstrated by FIG. 9A of the patent application drawings for this AMS_BioRhythms program) as requested by the user.
48-j) While the requested graphic is displayed, the user may, via the “Back” button begin the graphic at any earlier date or via the “Forward” button begin the graphic at any later date.
48-k) While the requested graphic is displayed, the user also has the option of entering an entirely new date to begin the graphic representation.
48-l) When finished with the on-screen graphic requested, the user may close the graphic window, returning to the bio-rhythm menu frame where the user may choose either a new graphic representation, return to the data entry frame, or close the program completely. Choosing to close the program will close all AMS BioRhythms computing program frames except for the on-screen icon.

1) This AMS_BioRhythms computing program is capable of being installed to be used in the home on any personal computer, laptop, tablet, phone, any computer server system for in office use or for use by customers or clients via the internet and/or any other computing device that allows input into such device/s for personal use and data entry so that any individual, or other entity, may know the bioryhmic implications of any entity's position, or connection, with any other entity or any other situation that any AMS_BioRhythms computing program user may wish to investigate via the graphic representations produced by this AMS_BioRhythms computing program.

2) Any Bio-Rhythm graphic/s and other information produced by this AMS_BioRhythms computing program, as claimed in “1)” above, are based upon the investigated entity's, or situation's, birthdate, inception date and/or incident date as defined by the user's input via the various data entry panels of this AMS_BioRhythms computing program.

3) This AMS BioRhythms computing program, as claimed in “1)” above, and the new graphic representations of each individual cycle graphic chart representation, as produced by this AMS_BioRhythms computing program, as claimed in “1)” above, have been solely, and independently, developed by Ronald L. Monarch as owner/operator of Astro Metaphysical Services.

4) Ronald L. Monarch as the inventor of this AMS BioRhythms computing program has given each of the “Physical”, “Emotional” and “Intellectual” cycle representations produced by this AMS_BioRhythms computing program, as claimed in “1)” above, their own more distinctive graphic representation and color since the single daily Bio-Rhythms graphics as previously promoted by Gittleson and others are poorly designed, poorly descriptive and fail to more properly describe Bio-Rhythm cycles and the incidents described by said Bio-Rhythm cycles.

5) With the correction and advancements to, and beyond that claimed in “4)” above, any user of this AMS_BioRhythms computing program will possess the ability to produce the appropriate appropriate Bio-Rhythm cycle graphic representations, as claimed in “1)”above, either on a daily cycles graphic or on a yearly cycles graphic for either any single entity or incident or for a combination of any two entities or incidents as a more appropriate graphic expression of such Bio-Rhythm cycles.

6) Therefore, the graphic representations, as claimed in “1)”, “4)” and “5)” above, are produced with a more distinct look and visual appearance so as to make any of the graphic Bio-Rhythms compilations produced by this AMS_BioRhythms computing program, as claimed in “1)” above, more easily and quickly decipherable and understood.

7) Beyond such as stated in claim “4)” above, and as has been put forth by this inventor, Ronald L. Monarch, and is herein described within claims “5)” and “6)” above, up to and including the date of the writing of this herein United States Patent Application for this AMS_BioRhythms computing program, there apparently fails to exist any evidence that any other individual, or entity, has proposed anything close to any innovative advancements to the study of Bio-Rhythm cycles.

8) Such failure, as claimed in “4)” and “7)” above, is even more true and relevant concerning the single yearly, double daily and double yearly Bio-Rhythms graphic representations since Ronald L. Monarch is the sole, and independent, developer and inventor of such additions to the idea of Bio-Rhythm cycles.

9) Any authorized user of this AMS_BioRhythms computing program will possess the ability to project any of the graphic representations produced by this AMS_BioRhythms computing program, as claimed in “1)”, “4)”, “5)”, “6)” and “7)” above, either forward to a future date or backward to a previous date for an easy wider ranging analysis of the entity's Bio-Rhythm cycles and such Bio-Rhythm cycles implications.

10) Ronald L. Monarch, as the inventor of this AMS_BioRhythms computing program, has found that this AMS_BioRhythms computing program is meant to, and indeed does, assist more efficiently and effectively, in the study of the manner in which Bio-Rhythm cycles actually affect the life time of any human, animal, business, country or any other entity or situation and the manner in which any such entity or situation is likely to interact with any other entity or situation.

11) With the consideration of the claims in “1)”, “4)” and “10)” above and the associated claims of each, Ronald L. Monarch has found, through the study of the Bio-Rhythm graphics produced by this AMS_BioRhythms computing program, as claimed in “1)” above, that this AMS_BioRhythms computing program can be used as a “Person of Interest” and/or “Pre-Crime” tool!

12) Bearing in mind the claims of “10)” and “11)” above, this inventor has seen, through the use of the graphic representations, as claimed in “1)” above, and study using this AMS_BioRhythms computing program, how this program can be used in the more specific and accurate questioning of any “suspect” in connection with any situation that said “suspect” may have been involved with.

13) As demonstrated via study of the accompanying graphic drawing examples of the screen output of this AMS_BioRhythms computing program, as claimed in “1)”, “5)” and “6)” above, one is able to see and determine the high level of accuracy of the Bio-Rhythm graphics charts produced by this AMS_BioRhythms computing program, as claimed in “1)”, “5)” and “6)” above, in their depiction of any entity's or situation's Bio-Rhythmic cycles for any specific date during the life cycle of any such entity or situation.
